SEQUENCE LISTING (13 sequences)

SEQ ID NO: 1 - DNA, 2382 bp, Human
  CDS (1)..(2214)
atgcactgtatgagcggccactccaattttgtatcttgtgtatgcatcataccctcaagtgacatctaccctcatggcctaattgccaccggtggaaatgaccacaatatatgcattttctcactggacagtccaatgccactttatattctaaaaggccacaaaaatactgtttgtagtctatcatctggaaaatttgggacattacttagtggttcatgggacaccactgctaaagtctggctgaatgacaagtgcatgatgaccttgcagggtcatacagctgcagtgtgggcggtaaagatcttacctgaacagggcttaatgttgactggatcagcagacaagactgttaaactgtggaaggctggaagatgtgagaggactttttcagggcatgaagactgtgtaagaggtttggcaattttgagtgaaacagaatttctttcctgtgcaaatgatgctagtattagaaggtggcaaatcactggcgagtgtcttgaagtatattatggacatacaaattatatttatagcatatccgtttttccaaattgtagagactttgtgacaacagcagaggacagatctctgagaatctggaaacatggggaatgtgctcaaactatccgacttccagctcagtctatatggtgctgctgtgtgctcgacaatggtgacattgtggttggtgcgagtgatggcattattagagtgtttacagagtcagaagatcgaacagcaagtgctgaagaaatcaaggcttttgaaaaagaactgtctcacgcaaccattgattctaaaactggcgatttaggggacatcaatgctgagcagcttcctgggagggaacatcttaatgaacctggtactagagaaggacagactcgtctaatcagagatggggagaaagtcgaagcctatcagtggagtgttagtgaagggaggtggataaaaattggtgatgttgttggctcatctggtgctaatcagcaaacatctggaaaagttttatatgaagggaaagaatttgattatgttttctcaattgatgtcaatgaaggtggaccatcatataaattgccatataataccagtgatgacccttggttaactgcatacaacttcttacagaagaatgatttgaatcctatgtttctggatcaagtagctaaatttattattgataacacaaaaggtcaaatgttgggacttgggaatcccagcttttcagatccatttacaggtggtggtcggtatgttccgggctcttcgggatcttctaacacactacccacagcagatccttttacaggtgctggtcgttatgtaccaggttctgcaagtatgggaactaccatggccggagttgatccatttacagggaatagtgcctaccgatcagctgcatctaaaacaatgaatatttatttccctaaaaaagaggctgtcacatttgaccaagcaaaccctacacaaatattaggtaaactgaaggaacttaatggaactgcacctgaagagaagaagttaactgaggatgacttgatacttcttgagaagatactgtctctaatatgtaatagttcttcagaaaaacccacagtccagcaacttcagattttgtggaaagctattaactgtcctgaagatattgtctttcctgcacttgacattcttcggttgtcaattaaacaccccagtgtgaatgagaacttctgcaatgaaaaggaaggggctcagttcagcagtcatcttatcaatcttctgaaccctaaaggaaagccagcaaaccagctgcttgctctcaggactttttgcaattgttttgttggccaggcaggacaaaaactcatgatgtcccagagggaatcactgatgtcccatgcaatagaactgaaatcagggagcaataagaacattcacattgctctggctacattggccctgaactattctgtttgttttcataaagaccataacattgaagggaaagcccaatgtttgtcactaattagcacaatcttggaagtagtacaagacctagaagccacttttagacttcttgtggctcttggaacacttatcagtgatgattcaaatgctgtacaattagccaagtctttaggtgttgattctcaaataaaaaagtattcctcagtatcagaaccagctaaagtaagtgaatgctgtagatttatcctaaatttgctgtagcagtggggaagagggacggatatttttaattgattagtgtttttttcctcacatttgacatgactgataacagataattaaaaaaagagaatacggtggattaagtaaaattttacatcttgtaaagtggtggggaggggaaacagaaataaaatttttgcactd

SEQ ID NO: 2 - protein, 738 aa, Human
MHCMSGHSNFVSCVCIIPSSDIYPHGLIATGGNDHNICIFSLDSPMPLYILKGHKNTVCSLSSGKFGTLLSGSWDTTAKVWLNDKCMMTLQGHTAAVWAVKILPEQGLMLTGSADKTVKLWKAGRCERTFSGHEDCVRGLAILSETEFLSCANDASIRRWQITGECLEVYYGHTNYIYSISVFPNCRDFVTTAEDRSLRIWKHGECAQTIRLPAQSIWCCCVLDNGDIVVGASDGIIRVFTESEDRTASAEEIKAFEKELSHATIDSKTGDLGDINAEQLPGREHLNEPGTREGQTRLIRDGEKVEAYQWSVSEGRWIKIGDVVGSSGANQQTSGKVLYEGKEFDYVFSIDVNEGGPSYKLPYNTSDDPWLTAYNFLQKNDLNPMFLDQVAKFIIDNTKGQMLGLGNPSFSDPFTGGGRYVPGSSGSSNTLPTADPFTGAGRYVPGSASMGTTMAGVDPFTGNSAYRSAASKTMNIYFPKKEAVTFDQANPTQILGKLKELNGTAPEEKKLTEDDLILLEKILSLICNSSSEKPTVQQLQILWKAINCPEDIVFPALDILRLSIKHPSVNENFCNEKEGAQFSSHLINLLNPKGKPANQLLALRTFCNCFVGQAGQKLMMSQRESLMSHAIELKSGSNKNIHIALATLALNYSVCFHKDHNIEGKAQCLSLISTILEVVQDLEATFRLLVALGTLISDDSNAVQLAKSLGVDSQIKKYSSVSEPAKVSECCRFILNLL

SEQ ID NO: 3 - protein, 26 aa, Human
GIGAVLKVLTTGLPALISWIKRKRQQ

SEQ ID NO: 4 - protein, 6 aa, Artificial Sequence
  Description of Artificial Sequence  Synthetic Peptide
KRKRQQ

SEQ ID NO: 5 - protein, 5 aa, Artificial Sequence
  Description of Artificial Sequence  Synthetic Peptide
KVLTT

SEQ ID NO: 6 - protein, 21 aa, Artificial Sequence
  Description of Artificial Sequence  Synthetic Peptide
ESPLIAKVLTTEPPIITPVRR

SEQ ID NO: 7 - protein, 14 aa, Artificial Sequence
  Description of Artificial Sequence  Synthetic Peptide
ESPLIAKVLTTEPP

SEQ ID NO: 8 - protein, 15 aa, Artificial Sequence
  Description of Artificial Sequence  Synthetic Peptide
KVLTTEPPIITPVRR

SEQ ID NO: 9 - protein, 21 aa, Artificial Sequence
  Description of Artificial Sequence  Synthetic Peptide
ESPLIALKKAALAAIITPVRR

SEQ ID NO: 10 - protein, 8 aa, Artificial Sequence
  Description of Artificial Sequence  Synthetic Peptide
KVLTTEPP

SEQ ID NO: 11 - protein, 21 aa, Artificial Sequence
  Description of Artificial Sequence  Synthetic Peptide
LAAKRPKVLTTEPPRRAAKII

SEQ ID NO: 12 - protein, 15 aa, Artificial Sequence
  Description of Artificial Sequence  Synthetic Peptide
NSKDFVTTSEDRSLR

SEQ ID NO: 13 - protein, 16 aa, Artificial Sequence
  Description of Artificial Sequence  Synthetic Peptide
RVFTESEERTASAEEI

(19) United States
(12) Patent Application Publication
(10) Pub. No.: US 2003/0166548 A1
(43) Pub. Date: Sep. 4, 2003

(54) PLAP polypeptides and methods of producing and using same

(75) Inventors: Johnny W. Peterson, Dickinson, TX (US); Shamsher S. Saini, Galveston, TX (US); Thomas G. Wood, Houston, TX (US); Ashok K. Chopra, Galveston, TX (US)

Correspondence Address: Mark B. Wilson, FULBRIGHT & JAWORSKI L.L.P., Suite 2400, 600 Congress Avenue, Austin, TX, 78701, US

(73) Assignee: Board of Regents, The University of Texas System, Austin, TX

(21) Appl. No.: 10/175,270

(22) Filed: Jun. 19, 2002

Related U.S. Application Data
(62) Division of application No. 09/458,397, filed on Dec. 10, 1999.
Application No. 09/458,397 is a continuation of application No. PCT/US98/12340, filed on Jun. 11, 1998.
(60) Provisional application No. 60/049,316, filed on Jun. 11, 1997.

(51) Int. Cl. A61K 38/17; G01N 33/53

(52) U.S. Cl. 514/12; 435/7.1

(57) ABSTRACT

Synthetic milittin and related peptides derived from the sequence of human phospholipase A₂-activating protein (PLAP), free from the contaminating phospholipase A₂ (PLA₂) and/or present when the peptides are purified from natural sources, show inhibitory activity against PLA₂. Inhibition of this enzyme, responsible for the hydrolysis of arachiodonate, an important precursor of eicosanoids, should lead to a decrease in the inflammatory response. In addition to their use as anti-inflammatory therapeutics, compositions containing the synthetic peptides may also be useful therapeutic tools for diagnosing inflammatory diseases (e.g., Crohn's disease, ulcerative colitis, rheumatoid arthritis).

This application claims priority from U.S. Serial No. 60/049,316, filed Jun. 11, 1997.

### BACKGROUND OF THE INVENTION

1. Field of the Invention

The present invention relates generally to the fields of diagnosing and treating inflammatory diseases. More particularly, it concerns the uses of synthetic peptide and peptide analog inhibitors of phospholipase A₂.

2. Description of Related Art

Inflammation is normally a basic host protective mechanism that increases resistance to microorganisms and other foreign antigens. Inflammatory responses are mediated in part by the eicosanoids, C₂₀ products of the cyclooxygenase and lipooxygenase metabolism of arachidonic acid. The eicosanoids include proinflammatory prostaglandins (e.g., PGE₂) and leukotrienes (e.g., LTB₄). The level of arachidonic acid, itself, is mediated by the action of phospholipase A₂ (PLA₂) a lipophilic enzyme that hydrolyzes arachidonic acid from the sn-2 position of phospholipids located in cell membranes. PLA₂ enzyme activity is increased by a eukaryotic cell protein referred to as phospholipase A₂-activating protein (PLAP), which was initially cloned from murine BC₃H₁ cells (Clark, 1991). Murine PLAP is reported to increase PLA₂ enzyme activity (Bomalaski et al, 1994).

Inflammation is also controlled by cytokines, which are proteins secreted by leukocytes and other eukaryotic cells. Along with eicosanoids, cytokines serve as signals to inflammatory cells, and extensive interplay between these mediators is responsible for orchestrating the inflammatory response.

Uncontrolled inflammation can cause extensive tissue damage and is the hallmark of numerous diseases, including rheumatoid arthritis; tuberculosis; asthma; burr, wounds; inflammatory diseases of the gastrointestinal tract, including Crohn's disease, Barrett's esophagus, pancreatitis, ulcerative colitis; and inflammatory infectious diseases such as salmonellosis, shigellosis, and amoebiasis. Of these diseases, rheumatoid arthritis alone affects one percent of all populations, including about 3 million Americans, and is generally characterized by inflammation of the small joints. The clinical course of the disease varies and diagnosis is sometimes difficult. In ten to fifteen percent of all patients, rheumatoid arthritis leads to severe deformities and crippling.

Many currently employed anti-inflammatory drugs, including aspirin, ibuprofen, and acetaminophen are directed at blocking the synthesis of eicosanoids from arachidonic acid via the cyclooxygenase and lipooxygenase pathways. Given the cytotoxic side effects associated with many of the current drugs, the large dosages required by many current therapies, and the huge potential market, the search for new anti-inflammatory drugs is an area of intense research.

Historically, some early patients with rheumatic diseases were treated with live bee stings. The first scientific medical report on apitherapy was published in 1859 by Demartis. Osol and Farrar in 1955 summarized the status of bee venom therapy for arthritis and related conditions. Since that report, apitherapy has been discussed extensively as a treatment for arthritis and other diseases by the lay public. A later scientific report on bee venom therapy for arthritis patients appeared in 1966 (Steigerwaldt et al.). More recently, Haberman (1972) reviewed his own research and that of others, comparing the pharmacological and biochemical activities of constituents of various animal venoms. Other reports on bee venom therapy for arthritis were performed using experimental animal models of arthritis [Zurier et al., 1973; Chang and Bliven, 1979; Eiseman et al., 1982; Somerfield, 1983, 1986a, 1986b; Somerfield et al., 1986; Somerfield and Brandwein, 1988; Tannenbaum and Greenspoon, 1982]. In 1993, Yiangou et al reported that bee venom injections markedly reduced paw edema in adjuvant arthritic rats

One major component of bee venom is melittin (MLT), a 26 amino acid peptide highly related to murine, rat, and human phospholipase A₂-activating proteins (PLAP). One recent study directed at the improved diagnosis of rheumatoid arthritis has centered on the detection of human PLAP [Bomalaski et al., U.S. Pat. No. 5,294,698]. In U.S Pat. No. 5,294,698, the inventors refer to “human PLAP”, but the supporting amino acid sequences shown in the application are derived from the murine Plap gene (Clark et al. 1991 and GenBank accession number M57958). The sequence published by Clark et al. (1991) for murine PLAP is approximately one half the size of human or rat PLAP. The inventors contend that the PLAP protein is an activator of PLA₂ and is induced during inflammatory responses, serving to enhance the activity of PLA₂ in inflamed tissues. Activation of endogenous PLA₂ in intact cells by PLAP-related melittin has also been suggested [Molay et al., 1976; Shier, 1979], and melittin has been used as a probe for stimulating endogenous PLA₂ activity by various investigators [Engelsen and Zatz, 1982; Pisano, 1983; Grandison, 1984; Salari et al., 1985; Okano et al, 1985; Abuou-Samra et al., 1986; Knepel and Gerhards, 1987; Rosenthal and Jones, 1988; Heisler, 1989; Liu and Jackson, 1989].

Most of these studies used commercial melittin, isolated from bee venom, which has been reported to be contaminated with heat-stable PLA₂. Bee venom PLA₂ contamination of purified natural melittin has been reported to increase tissue PLA₂ activity by as much as 75% Fletcher et al., 1990]. This observation may explain why melittin has appeared to enhance PLA₂ activity [Metz, 1986]. However, some investigators have reported that the purified natural melittin preparations used in their studies did not have any detectable PLA₂ contamination [Shier, 1979; Whistler, 1989]. There are recent reports of enhanced PLA₂ activity in a unilamellar system with a highly purified preparation of melittin [Rao, 1992], as well as in PC12 cells, by synthetic melittin [Choi et al., 1992]. To some extent, PLA₂ activity is dependent on whether liposomes, containing the phospholipid substrate, have been sonicated [Mollay and Kreil, 1974] or used nonsonicated [Yunes et al., 1977]. The mechanism of enhanced PLA₂ activity could therefore be related to alteration in substrate availability [Dufourcq et al., 1986]Alternatively, the reported stimulation of PLA₂ activity could be explained by melittin's stimulatory effect on phospholipase D (PLD). Recently, we observed that addition of synthetic melittin to dissociated membranes from a human monocyte cell line (U-937), resulted in the formation of phosphatidic acid and release of arachidosic acid. The latter step is made possible by the action of a second enzyme, diacylglycerol kinase. PLAP may also exert a stimulatory effect on PLA₂ because of the type of PLA₂ selected. Some types of PLA₂ are considered cytosolic (e.g., 85 kDa), while others are secreted (eg., 14 kDa) from cells. PLA₂ present in synovial fluids is largely low molecular weight secretory PLA₂ and is inhibited by synthetic melittin (Table 1). Melittin also inhibits human serum PLA₂ purified on a melittin-Sepharose column (FIG. 7 and Table 3).

### SUMMARY OF THE INVENTION

The present invention is directed toward the use of novel synthetic melittin and synthetic PLAP-like peptides or analogs such as peptide G22R, peptide G23, peptide G24, peptide G25, peptide G26, peptide G27, peptide G141, peptide G142, peptide F31, or peptide F32, e.g., as anti-inflammatory drugs. These peptides and similar ones are inhibitors of PLA₂, the enzyme responsible for hydrolyzing arachidonic acid from phospholipids. Arachidonic acid is the precursor of eicosanoids, the compounds thought to mediate inflammation. Melittin and the related PLAP peptides are currently thought to be PLA₂ activators, however, studies described herein aimed at determining the effect of both purified natural and synthetic melittin on PLA₂ have clearly indicated that natural melittin, as suspected, is contaminated with a heat-stable PLA₂, which is responsible for giving the appearance of enhancing PLA₂ activity. Synthetic melittin, on the other hand, as well as synthetic peptides made to regions of murine PLAP, bind to and inhibit several types of PLA₂ activity in cell-free assays and inhibit the several types of PLA₂ activity of cultured cells in vitro (Saini et al. 1997).

Some patients do not respond to current anti-inflammatory drugs. Moreover, anti-inflammatory medications currently in use oftentimes have undesirable side effects or require large dosages. For example, steroids tend to inhibit the overall function of the immune response and long term use is not recommended Many other medications, including aspirin can irritate the gastrointestinal tract. Other drugs, including ibuprofen have cytotoxic side effects and may cause liver damage Medications based on synthetic melittin or related peptides have potentially fewer side effects and would be degraded in the patient into normal amino acid metabolites.

The invention also relates to the use of these peptides for the affinity chromatography and quantification of PLA₂. Such in vitro methods for determining the presence of PLA₂ could provide useful assays for the diagnosis of inflammatory bowel diseases which are characterized by increased plasma levels of PLA₂. Purified PLA₂ and antibodies to the PLAP-like peptides may also be useful for the purification of the PLAP protein, which is present along with PLA₂ in the synovial fluid of arthritis patients and in intestinal tissues of patients with Crohn's Disease and ulcerative colitis. The complete sequence of human PLAP is included in this application and its unique partial sequences will be useful in diagnosis and treatment of several inflammatory diseases.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F shows the cDNA sequence (SEQ ID NO. 1) and resultant amino acid sequence (SEQ ID NO. 2) of human PLAP.

FIG. 1G shows sequences of melittin and the synthetic peptides of the present invention.

FIG. 2 shows the effect of synthetic melittin on the release of [³H]oleic acid from [³H]-oleic acid-labeled E. coli. The curves depicted by closed circles and squares illustrate spontaneous release of ³H-oleic acid from the bacterial cells by termination of the assay with HCl. This procedure could mimic enzymatic activity, both at 37° C. and 0° C., a condition incompatible with PLA₂ enzyme activity. The closed triangles denote the lack of enzymatic activity at 0° C./15 min. when the reaction was terminated with cold PBS.

FIG. 3 shows the molar concentration of synthetic melittin required for maximal inhibition of bee venom PLA₂ mediated release of [³H]-oleic acid from [³H]-oleic acid-labeled E. coli. PLA₂ concentration was held constant at 1 nM. The amount of synthetic melittin required to inhibit maximal PLA₂ activity (80%) is 30 mM.

FIG. 4 shows a Lineweaver-Burk plot of bee venom PLA₂ activity with [¹⁴C]-arachidonic acid-labeled phosphatidylcholine substrate in the presence and the absence of synthetic melittin. KM is both cases is 0.75 nM, while Vₘₐₓ decreased from 200 to 50 μmoles/min/mg protein.

FIG. 5 shows the inhibitory effect of the various synthetic PLAP-like peptides on [³H]-arachidonic acid metabolite release from murine monocyte/macrophage J774 cells in the presence and absence of cholera toxin (CT). FIG. SA shows the inhibitory effect of synthetic peptide G22R. FIG. 5B shows the effect of G23, FIG. 5C the effect of G24, FIG. 5D the effect of G26, FIG. 5E the effect of G25, and FIG. 5F the effect of G27.

FIG. 6 shows the results of enzyme-linked microtiter assays of synthetic peptide-PLA₂ binding. FIG. 6A shows binding of G22B to a plate coated with purified PLA₂. FIG. 6B shows the interference of increasing dosages of synovial fluid PLA₂ to streptavidin-biotin binding on a biotinylated melittin coated plate.

FIG. 7. Chromatography of human serum on peptide—Sepharose columns: 10 ml of heat inactivated human serum was diluted to 1:10 with PBS and loaded onto Melittin-Sepharose column (●) and PLAP—Sepharose column (▪) pre-equilibrated with PBS. Columns were washed with 500 ml of PBS for overnight and eluted with 0.5M glycine/HCl buffer, pH 2.8.

### DESCRIPTION OF ILLUSTRATIVE EMBODIMENTS

One broad aspect of the present invention is directed to providing novel PLA₂ inhibitors, including synthetic melittin and related compounds. In a preferred embodiment of this aspect of the invention, these compounds will be used as a new class of anti-inflammatory drugs, particularly as therapies or prophylaxes for diseases with inflammatory effects such as rheumatoid arthritis, for example.

In a second broad aspect of the invention, the synthetic peptides of the invention can be used as diagnostic tools for detecting the amount of PLA₂ released into body fluids during inflammatory diseases. In a preferred embodiment of this second aspect of the invention, synthetic melittin and related PLAP-like peptides are used for the affinity-purification and quantitation or detection of PLA₂. The methods for the purification and quantification of PLA₂ of this invention are useful as novel diagnostic tools in the detection of inflammatory diseases, especially rheumatoid arthritis and inflammatory bowel diseases Further, antibodies to the unique amino acid sequences of human PLAP will be useful diagnostic reagents.

Inflammation is normally a protective cellular response orchestrated by proinflammatory eicosanoids (e.g., PGE₂ and LTB₄) and cytokines (e.g., TNFα and IL-8). In contrast, excessive production of these mediators is integral to many diseases, such as rheumatoid arthritis, asthma, Crohn's disease, ulcerative colitis, and bacterial infections. Because of the stimulatory interplay among eicosanoids and cytokines, that inflammation can be modulated by reducing the synthesis and/or activity of eicosanoids These studies suggest that control of inflammation can be achieved by selective inhibition of PLA₂ activity with peptide analogs of PLA₂-activating protein (PLAP), which, in turn, reduce the release of the substrate (arachidonic acid) for eicosanoid biosynthesis. Studies described herein show that PLAP peptide analogs inhibit PLA₂ activity in cell-free enzyme assays and in cultured murine monocytes. Inflammation is further reduced by the administration of imidazole compounds (e.g., histidine) that protect tissue (Peterson et al., 1998) and reduce the biological activity of eicosanoids resulting from a covalent bond formed between the eicosanoids and imidazole. Objects of the present invention include: 1) identification and usage of PLAP peptide analogs inhibiting PLA₂ and eicosanoid synthesis in mononuclear and polymorphonuclear cells, 2) using PLAP peptide analogs to modulate PLA₂ and eicosanoid activity in vivo with concomitant reduction in cellular inflammation and pathology in animal models of inflammation, and 3) optimizing protection against inflammation-induced tissue injury conferred by imidazole-containing compounds administered alone and in combination with PLAP peptide analogs. Several inflammatory diseases should be controlled by therapeutic regulation of PLA₂ enzyme activity with synthetic PLAP peptide analogs and/or downregulation of proinflammatory eicosanoid activity by administration of imidazole derivatives.

1. Synthesis of PLA₂-Free Melittin and Related PLAP-Like Peptides

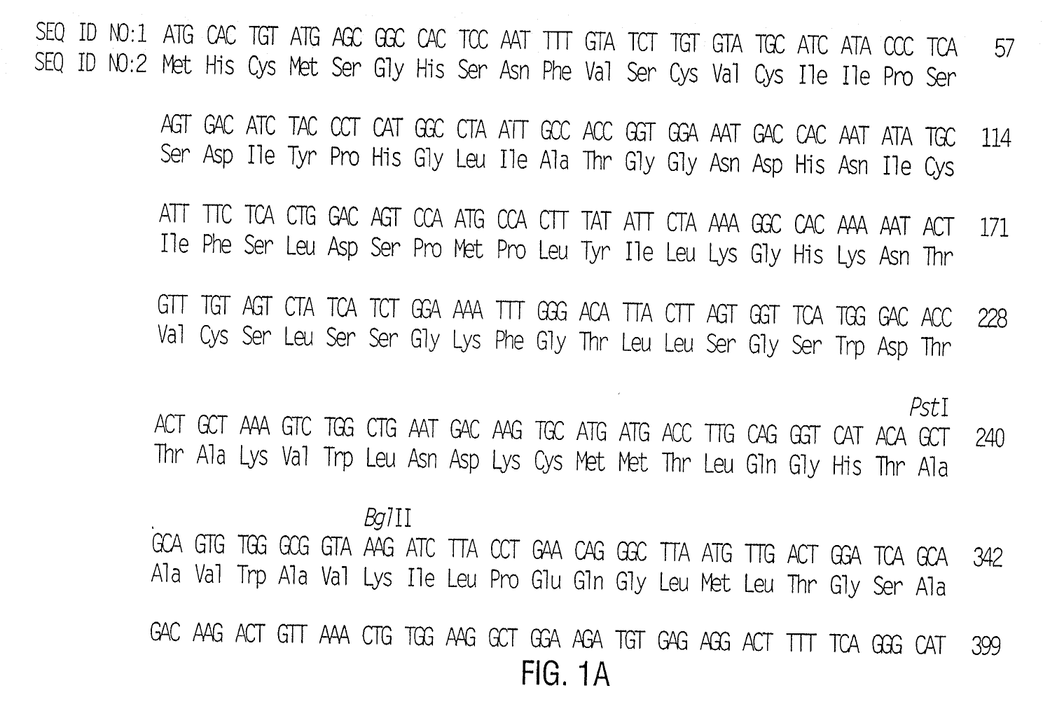

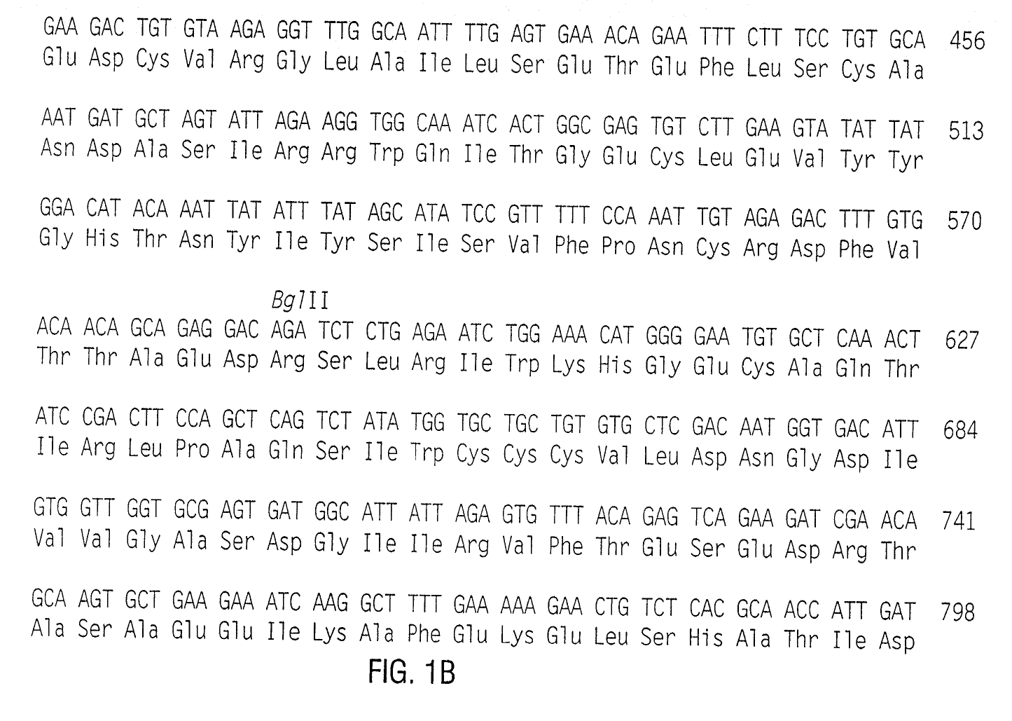

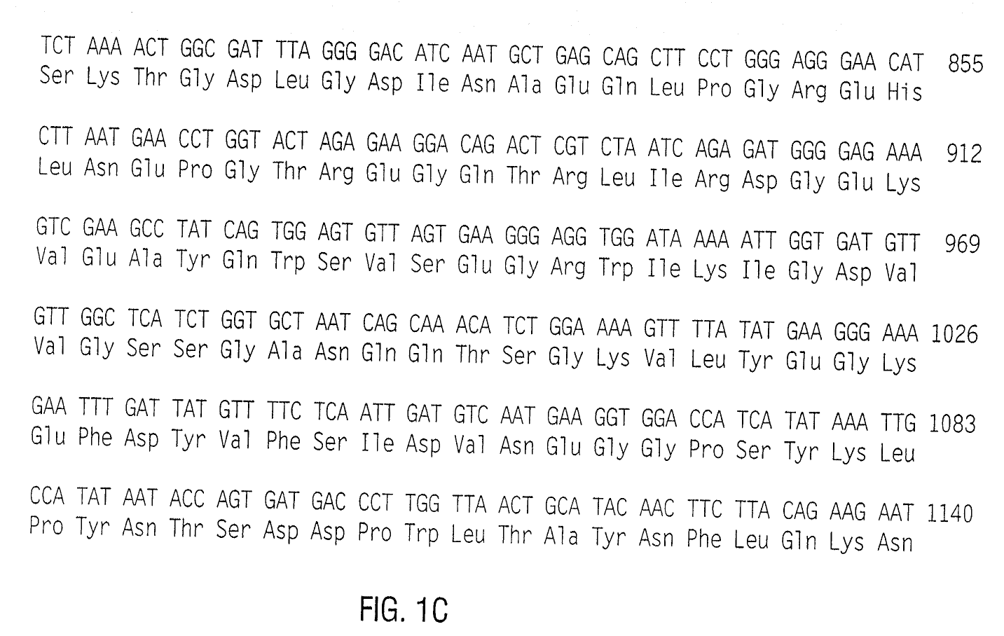

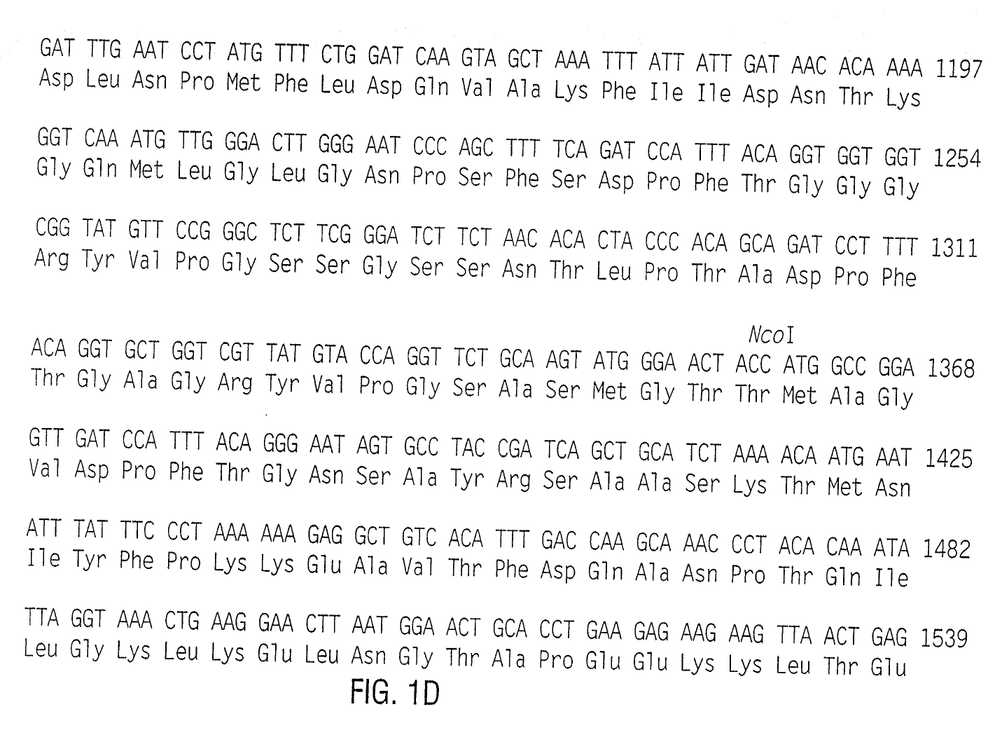

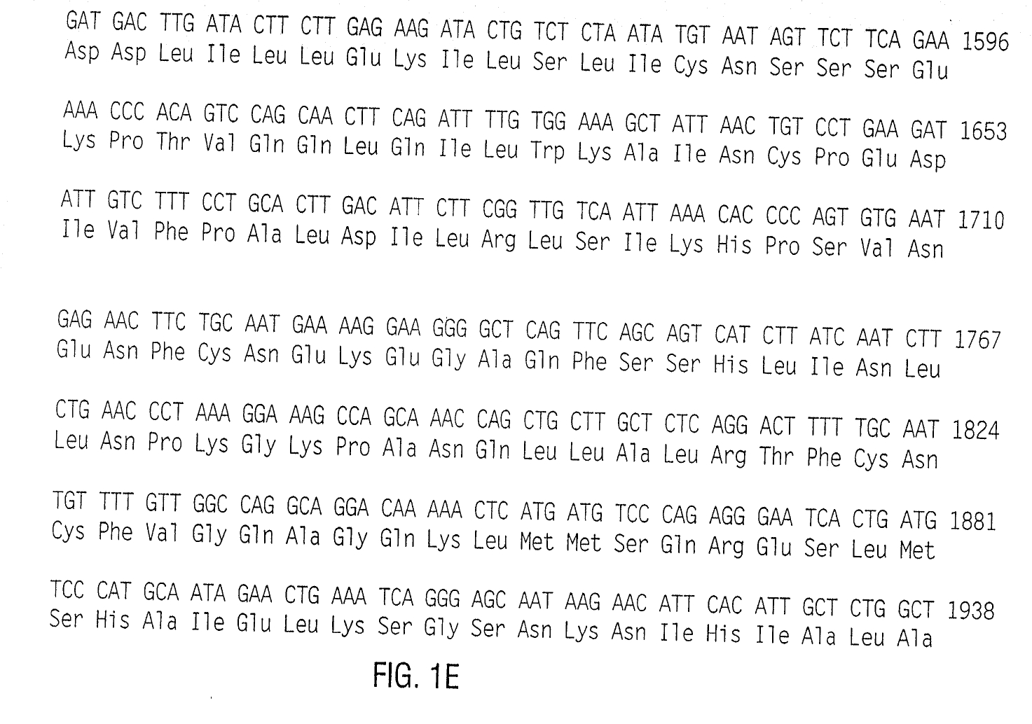

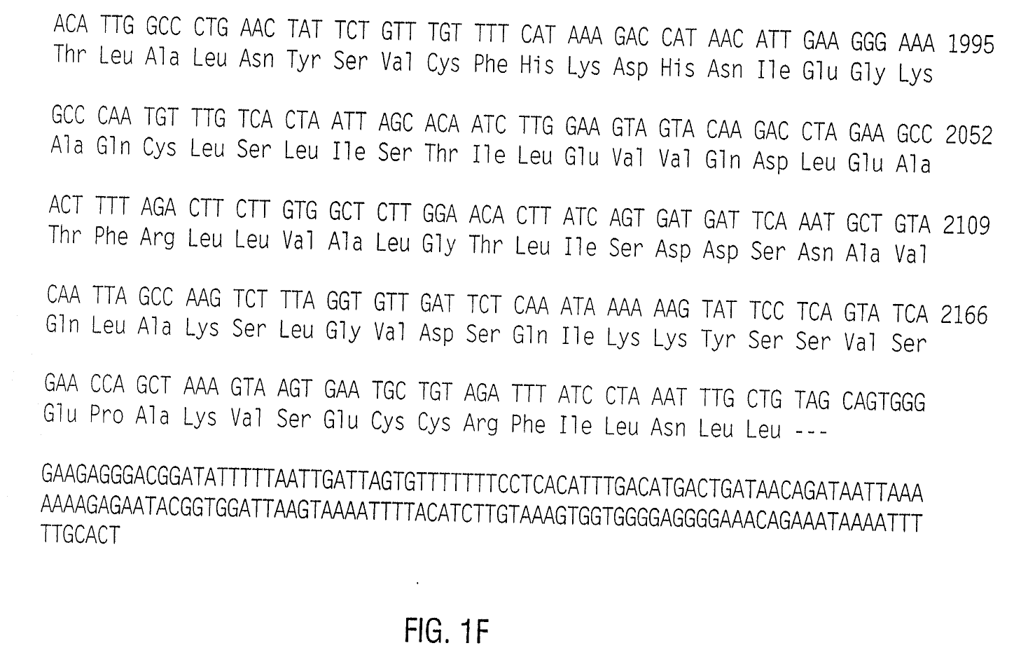

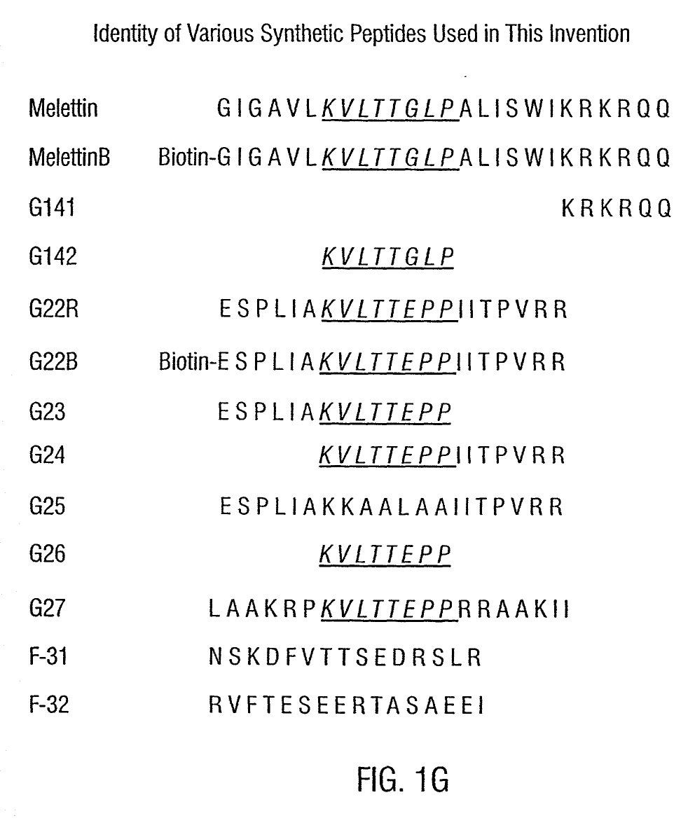

DNA sequences are available for both the murine and rat plap genes [Wang et al., 1995] One region of the murine PLAP protein (amino acids 260-280) is similar to that of melittin [Clark et al., 1991]. Given this information, the inventors envisioned the synthesis of a family of synthetic peptides based on the known sequences of natural melittin and the PLAP peptides from higher animals Methods of synthesizing a peptide of a given sequence are well known to those skilled in the art. For example, solid phase synthesis [Erickson, B. W. and Merrifield, R. B. in The Proteins (3rd Ed.) Vol. 2, pp 255-257, Academic Press (1977)] is useful for the preparation of short peptide sequences. The amino acid sequences of melittin and the related PLAP-like peptides that are part of this invention are shown in FIG. 1G. The sequence of the human plap gene also is available as described herein to generate additional synthetic peptides and/or oligonucleotides.

2. Complete DNA Sequence of Human PLAP

Using the technique of Reverse-Transcriptase Polymerase Chain Reaction (RT-PCR™) and 3′-Rapid Analysis of cDNA Ends (3′-RACE), the inventors cloned the full length human plap gene and determined the nucleotide sequence of the gene. The cDNA fragment contained 2381 nucleotides with a 738 codon open reading frame (2214 nucleotides) and should encode a protein having a molecular mass of 80,826 daltons. A comparison of the human plop cDNA sequence (see FIG. 1A-FIG. 1F and SEQ ID NO: 1) with the published sequence of the rat plap cDNA sequence shows 45 codons that differ in the first or second position and 138 codons that differ in the third position. (see FIG. 1A-FIG. 1F and SEQ ID NO: 1 )

3. Inhibition of PLA₂ by Melittin and Related Compounds

Evidence for the inhibition of PLA₂ activity can be shown using a number of assay systems. Some of the easiest methods of ascertaining the inhibitory effects of a given compound of this invention involve the use of cell-free PLA₂ enzyme assays. Methods of isolating PLA₂ from bee venom, snake venom and bovine pancreas have been previously described (Stefanski et al., 1986; Verheij et al., 1981; Hazlett and Dennis, 1985; Kortesuo et al, 1993).

In general, assays involve incubation of purified PLA₂ or a mixture believed to contain PLA₂, such as synovial fluid, with a PLA₂ substrate containing radio-labeled arachidonic acid in the presence of the inhibitor of interest. Suitable substrates for PLA₂ reactions include any glycerol-3-phosphate with arachidonic acid substituted at the sn-2 position. The preferred substrates of the assays of this invention include [¹⁴C]-arachidonic acid-substituted phosphatidylcholine or [¹⁴C]-arachidonic acid-substituted phosphatidylethanolamine. After the reaction is stopped by addition of chloroform and methanol, TLC chromatography is used to separate the released [¹⁴C] arachidonic acid from [¹⁴C] arachidonic acid-labeled phosphatidylcholine/ethanolamine, which may be quantified by scintillation counting.

PLA₂ activity may also be assayed using [³H]-oleic acid labeled Escherichia coli, in a modification of the procedure previously described by [Elsbach and Weiss, 1991]. E. coli cells grown in the presence of [³H]-oleic acid are incubated with PLA₂ enzyme. The reaction is quenched either by addition of 1M HCl or by dilution in cold phosphate buffered saline (PBS). The amount of [³H]-oleic acid released from the E. coli cells by PLA₂ is measured using scintillation counting.

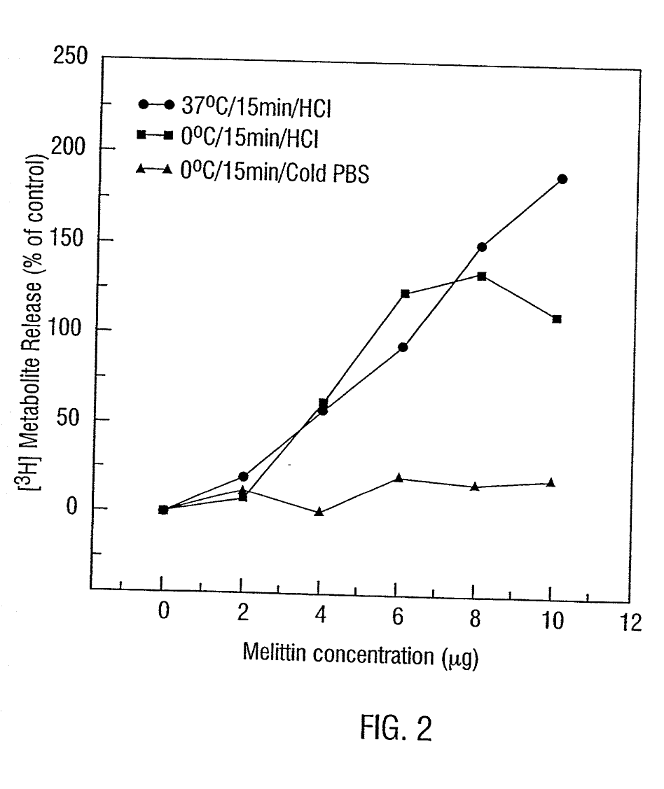

The preferred method for quenching the reaction is by dilution with cold PBS. As shown in FIG. 2, when reaction mixtures were incubated at 0° C. and cold PBS was used to quench the reaction, no release of [³H]-oleic acid was observed as the concentration of melittin was increased. In contrast, a melittin-dependent increase in the release of [³H]-oleic acid was observed when HCl was used to stop the reaction. This dose-dependent increase occurred at reaction temperatures from 0° C. to 37° C. This result indicates that melittin interacts with the phospholipids in the E. coli cell membrane and release of the complex from the bacterial cell surface is mediated by HCl. The fact that this phenomenon occurred at 0° C. indicates that this process does not involve an enzymatic process. This observation could further explain prior observations of melittin-enhanced PLA₂ activity in assay systems using acid quenching.

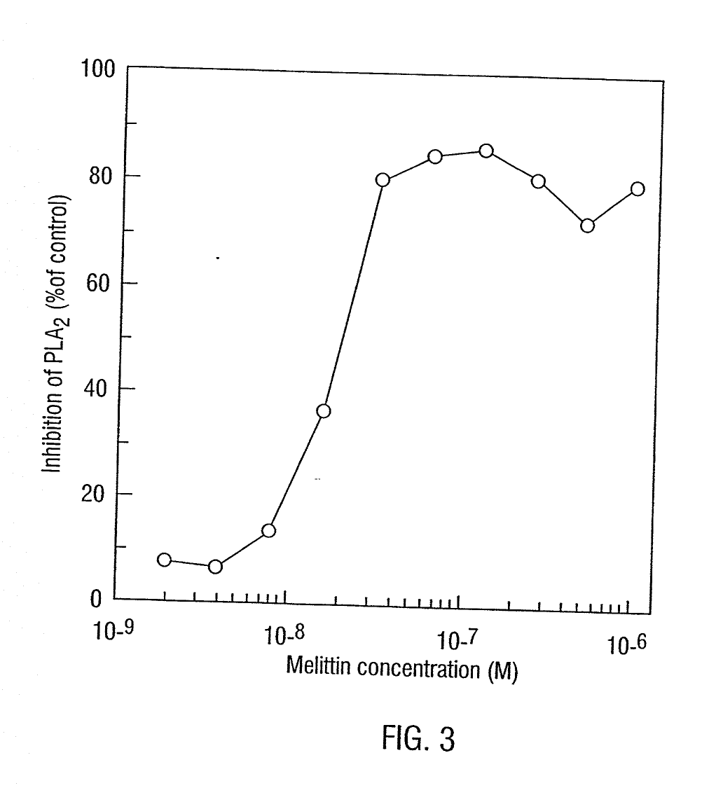

The results of the assays using synthetic melittin and other synthetic peptides related to PLAP are shown in Tables 1 and 2. The data confirm that synthetic melittin is the most potent synthetic inhibitor of PLA₂ activity. FIG. 3 shows the inhibition of 1 nM bee venom PLA₂ with various amounts of synthetic melittin. The synthetic melittin interacts with the bee venom PLA₂ in an approximately 30:1 molar ratio, causing 80% inhibition of PLA₂ enzyme activity. The amount of melittin required to cause approximately 50% inhibition of PLA₂ activity in vitro was very low (ED₅₀=2×10⁻⁸ M).

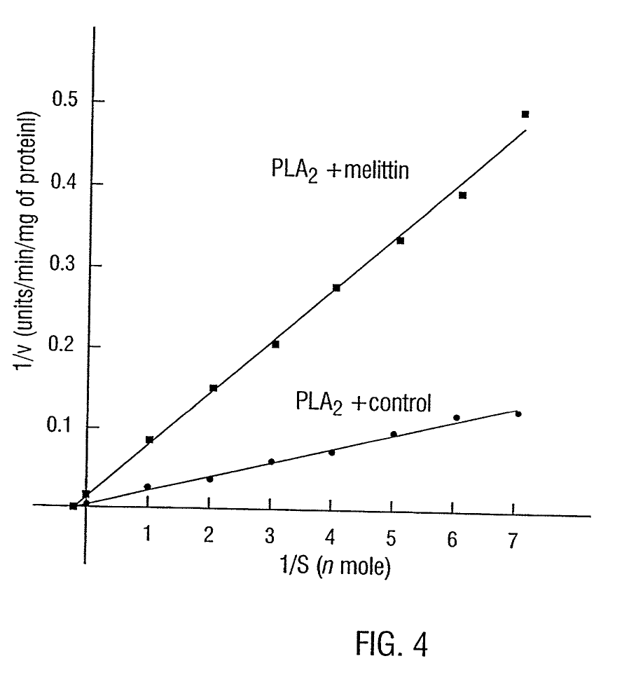

Lineweaver-Burk analysis (shown in FIG. 4) further characterizes melittin as a noncompetitive inhibitor of bee venom PLA₂ activity Melittin binds to the PLA₂ enzyme at a site other than the catalytic domain. The Kₘ in both the presence or absence of melittin remains the same (0.75 nM); however, the Vₘₐₓ of the enzyme (200 μM/min/mL) is decreased in the presence of melittin (50 μM/min/mL); [Siegel 1976; Dixon 1964].

4. Therapeutic Use of Melittin and Related Compounds as Anti-Inflammatory Drugs.

Inhibition of PLA₂ activity should result in decreased levels of arachidonic acid and subsequently to decreased levels of the biosynthetic products of arachidonic acid, the eicosanoid mediators of the inflammation response. Although synthetic melittin is a potent inhibitor of PLA2 and should provide beneficial effects as an anti-inflammatory compound, we have determined that melittin stimulates PLD, another phospholipase. By altering selected amino acids in the protein sequence of melittin, it should be possible to eliminate melittin's stimulatory effect on PLD. With this modification, melittin should be effective in clinical management of a variety of inflammatory diseases. Inhibiting PLA₂, therefore, should reduce inflammation. Patients with any of several inflammatory diseases would benefit from the therapeutic effects of having a medication that would reduce inflammation. Specific applications would include patients with rheumatoid arthritis, asthma, cardiovascular diseases, inflammatory diseases of the gastrointestinal tract, infectious inflammatory diseases and bum wound patients.

As used herein, “pharmaceutically acceptable carrier” includes any and all solvents, dispersion media, coatings, antibacterial and antifungal agents, isotonic agents and the like. The use of such media and agents for pharmaceutically active substances is well known in the art. Except insofar as any conventional media or agent is incompatible with the active ingredient, its use in the therapeutic composition is contemplated. Supplementary active ingredients can also be incorporated into the compositions. The phrase “pharmaceutically acceptable” also refers to molecular entities and compositions that do not produce an allergic or other untoward reaction when administered to an animal or a human.

Pharmacologically active compositions of the peptides would preferably be introduced directly to the inflamed tissues as parenteral preparations. The pharmaceutical preparations of synthetic melittin and the related PLAP-like peptides suitable for injectable use include sterile aqueous solutions or dispersions and sterile powders for the preparation of sterile injectable solutions or dispersions. In all cases the form must be sterile and must be of a viscosity suitable for syringability. It should be stable under the conditions of manufacture and storage and is preferably preserved against the contaminating action of microorganisms, such as bacteria and fungi. The carrier may be a solvent or dispersion medium containing, for example, water, ethanol, polyol (for example, gycerol, propylene glycol, and liquid polyethylene glycol, and the like), suitable mixtures thereof, and vegetable oils. The prevention of the action of microorganisms can be brought about by various antibacterial and antifungal agents, for example, parabens, chlorobutanol, phenol, sorbic acid, thimerosal, and the like. In most cases, it is preferable to include isotonic agents, for example, sugars or sodium chloride. An excipient such as serum albumin may be added to promote stability.

Sterile injectable solutions are prepared by incorporation of the active synthetic peptide or analogs in the required amount in the appropriate solvent with various other ingredients enumerated above, as required, followed by filter sterilization. Generally, dispersions are prepared by incorporating the various sterilized active ingredients into a sterile vehicle which contains the basic dispersion medium and the required other ingredients into a sterile vehicle which contains the basic dispersion medium and the required other ingredients from those enumerated above In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For parenteral administration in an aqueous solution, for example, the solution should be suitably buffered as necessary for the stability of the synthetic peptide or analog active ingredient and the liquid diluent first rendered isotonic with sufficient saline or glucose. Preferred pH range of the solution will be between 6.5 and 7.5. These particular aqueous solutions are especially suitable for intraperitoneal administration. In this connection, sterile aqueous media which can be employed will be known to those of skill in the art in light of the present disclosure.

The preferred dosage of synthetic peptide in a parenteral administration will vary, depending upon the size of the inflamed area, the severity of the symptoms associated with the inflammation and patient age, weight and medical history. The number of administrations of the parenteral composition will also vary according to the response of the individual patient to the treatment. In one exemplary application, the dosage of melittin-related analogs for treating inflammatory diseases would vary with the type of disease and the route of administration Further studies with animal models of inflammation to completely define projected doses.

In other preferred embodiments of the invention, pharmacologically active compositions could be introduced to the inflamed tissues through transdermal delivery of a medicated application such as an ointment, paste, cream or powder. Ointments include all oleaginous, absorption, emulsion and water-solubly based compositions for topical application, while creams and lotions are those compositions that include an emulsion base only Topically administered medications may contain a penetration enhancer to facilitate absorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives or other components desirable to preserve the active ingredient and provide for a homogenous mixture.

Suitable amounts of active ingredient synthetic peptide or analogs for compositions for topical administration typically may range from 0.1-10 μg peptide per 1000 g of composition. Administration of the ointments, creams and lotions of this invention may be from between once a day to as often as is necessary to relieve the symptoms of inflammation and will vary according to the strength of the medication, active ingredient, patient age and the severity of the symptoms. Administration of the topical medications of this invention will be applied directly to the inflamed area.

Another preferred method of administering pharmacologically active compositions of synthetic melittin analogs is as an aerosol. Aerosol compositions of the synthetic peptides of the present invention will be especially useful for the treatment of asthma, although they could also be used for dermal applications. The term aerosol refers to a colloidal system of finely divided solid of liquid particles dispersed in a liquified or pressurized gas propellant. The typical aerosol of the present invention for oral or nasal inhalation will consist of a suspension of active ingredients in liquid propellant or a mixture of liquid propellant and a suitable solvent Suitable propellants include hydrocarbons and hydrocarbon ethers Suitable containers will vary according to the pressure requirements of the propellant. Aerosols of this invention may contain 0.01-0.1 μg active ingredient. Administration of the aerosol will vary according to patient age, weight and the severity and response of the symptoms.

5. Quantification and Purification of PLA₂ with Melittin and Related PLAP-like Peptides.

In a further embodiment of the invention, an enzyme-linked microplate assay was developed to quantify PLA₂ from bee venom and synovial fluid. Enzyme-linked assays are well known in the art, and many assay formats may be found in standard texts [e.g. Harlow and Lane, Antibodies, A Laboratory Manual, Cold Spring Harbor Laboratory, Cold Spring Harbor, N.Y., 1988]. The assays are directed at detecting binding of microtiter plate-bound inhibitor to solution phase enzyme or microtiter plate bound enzyme to solution-phase inhibitor. One of these molecules, the inhibitor or the enzyme is conjugated to a detectable label or a molecule capable of complexing to a detectable label. Suitable detectable labels include horseradish peroxidase (HPRT), alkaline phosphatases, radiolabels such as ¹²³I and ³²P, biotin systems, latex particles, electron dense materials such as ferritin, fluorescent labels, or light scattering materials such as gold. Suitable methods of detecting the label include scintillation counting, autoradiography, fluorescence measurement, calorimetric measurement or light emission measurement.

A preferred labeling method of the assays of the present invention are biotin-avidin systems in which the inhibitor molecule of interest is biotin labeled. Addition of streptavidin-peroxidase allows for detection of peptide when streptavidin binds to the biotin of the bound peptide and a suitable substrate for peroxidase is added, resulting in a colored product. A suitable substrate for peroxidase is 2,2′-azinobis(3-ethylbenzthiazoline)sulfonic acid (ABTS). The preferred inhibitors for these assays include biotinylated synthetic melittin and the biotinylated derivatives of the other synthetic peptides shown in FIG. 1A-FIG. 1F. The most preferred inhibitors are biotinylated synthetic melittin and PLAP-peptide G22B (the biotinylated derivative of G22R). The enzyme of the inhibitor-enzyme complex used in the assays is PLA₂, either purified from an animal venom or present in a known or unknown concentration in samples of mammalian synovial fluid or serum In the most preferred embodiment of this aspect of the invention, the enzyme will be present in unknown concentration in a sample of human synovial fluid or serum.

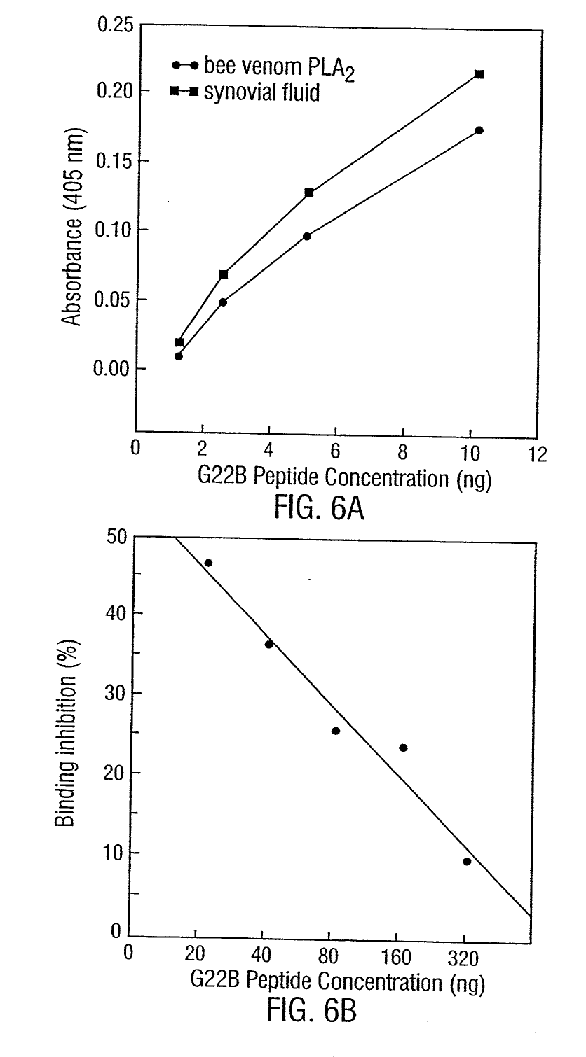

In an assay illustrating one preferred embodiment of this invention, biotinylated PLAP peptides are exposed to microtiter plates coated with either purified bee venom PLA₂ or synovial fluid from rheumatoid arthritis patients. A streptavidin-peroxidase conjugate is then used to detect biotinylated G22R bound to the PLA₂ on the plate. FIG. 6A indicates that the biotinylated PLAP-like peptide G22R directly binds to PLA₂ Components on the plates. In an example of another preferred embodiment of the invention, FIG. 6B shows the results of an experiment in which all wells of the plates were coated with biotinylated synthetic melittin. After blocking the wells with 10% bovine serum albumin, dilutions of synovial fluid from rheumatoid arthritis patients were added to the wells. Then, streptavidin-peroxidase was added. The graph shows evidence of a dose-dependent interference phenomenon suggesting that binding of synovial fluid PLA₂ to the biotinylated PLAP sterically hinders subsequent binding of the streptavidin-peroxidase reagent Further preferred embodiments of the invention relate to is the use of microtiter plates for the quantification of PLA₂ in the synovial fluid or serum of patients with inflammatory disease and the detection of rheumatoid arthritis.

The complete human plap DNA and amino acid sequence provided in this patent application will be useful in establishing specific reagents for use in diagnosis of inflammatory diseases. For example, Peterson et al. (1996) prepared antibodies to a synthetic peptide corresponding to murine PLAP (as residues 1 85-199) based on the amino acid sequence for murine PLAP reported by Clark et al. (1991). Although, human PLAP shows extensive homology with murine and rat PLAP, it possesses unique sequences that will be helpful in designing highly specific diagnostic reagents for clinical applications.

Another use of the PLA₂ binding capacity of the PLAP-like peptides is the purification of PLA₂ from animal venom, animal synovial fluid, or animal serum. In a preferred embodiment of the invention, melittin or PLAP-like peptides will be conjugated to Sepharose to purify PLA₂ from the synovial fluid of rheumatoid arthritis patient. Other solid phase support materials and methods of conjugating peptides to them are well known in the art [see for example Dean, P. D. G., Johnson, W. S., and Middle, F. A. (Eds.) Affinity Chromatography A Practical Approach, IRL Press (1985); Lowe, C. R. in Laboratory Techniques In Biochemistry and Molecular Biology, Vol. 7, Part 11, North-Holland (1979); Porath, J. and Kristiansen, T. in The Proteins (3rd Ed.), Vol 1, pp. 95-178, Academic (1975); Schott, H. Affinity Chromatography, Dekker (1984)].

### EXAMPLE 1

Peptide Synthesis

The peptides of the present invention were synthesized using a Vega Biotechnology Coupler 250. All peptides were analyzed for purity by high performance liquid chromatography using a C18 reverse-phase column. All peptides were stored as a dry powder at 4° C. until hydrated and then they were stored at −70° C. Some peptides were analyzed by mass spectrometry or amino acid analysis to confirm their identity.

Sequences of the peptides of this invention are shown in FIG. 1G and include SEQ ID No. 3, GIGAVLKVLTTGLPALISWIKRKRQQ; SEQ ID No. 4, KRKRQQ; SEQ ID No. 5, KVLTT; SEQ ID No. 6, ESPLIAKVLTTEPPIITPVRR; SEQ ID No. 7, ESPLIAKVLTTEPP; SEQ. ID No. 8, KVLTTEPPIITPVRR; SEQ ID No. 9, ESPLIALKKAALAAIITPVRR; SEQ ID No. 10, KVLTTEPP; SEQ ID No. 11, LAAKRPKVLTTEPPRRAAKII; SEQ ID No. 12, NSKDFVTTSEDRSLR; and SEQ ID No. 13, RVFTESEERTASAEEI.

### EXAMPLE 2

PLA₂ Assay Using Unsonicated Vesicles

Radiolabeled phosphatidylcholine or phosphatidylethanolamine containing [¹⁴C]-arachidonic acid in the sn-2 position of the phospholipid was dried under nitrogen and the lipid vesicles were prepared in 0.05% Triton X-100. The reaction mixture contained 10 μl of phospholipid substrate, 10 μl of 5× assay buffer (500 mM Tris[hydroxymethyl]aminomethane benzoate-HCl (Tris HCl), pH 8.0; 500 mM NaCl; 5.0% fatty acid-free bovine serum albumin (BSA); and 5 mM CaCl₂), 10 μl PLA₂ enzyme preparation, and sufficient H₂O to adjust the total volume to 50 μl The reaction mixture was incubated for 30 minutes at 37° C. in a waterbath and stopped by the addition of 50 μl of chloroform and methanol (1:3), containing 200 μg/ml of unlabeled arachidonic acid and then the mixture was vortexed Lipid was extracted by adding 50 μl of chloroform and 50 μl of 4M KCl, after which, the mixture was vortexed and centrifuged in a microfuge at 14,000 rpm for 10 minutes A 25 μl aliquot of the organic phase was spotted onto glass silica gel plates (Whatman LK6DF) and placed in a thin layer chromatography chamber with a solvent system containing petroleum ether, diethyl ether, and acetic acid (5:25:1) for 30 minutes. The lipid spots were made visible by placing the silica gel plates in a chamber containing iodine vapor. The silica gel region corresponding to arachidonic acid was scraped and transferred to scintillation vials containing 10 ml of scintillation cocktail, which is commercially available and designed to measure the amount of radioactivity in aqueous samples when used with a liquid scintillation counter. The amount of radionuclide corresponding to [¹⁴C]-arachidonic acid in each vial was determined in a Beckman liquid scintillation counter. Results of these assays are shown in Tables 1 and 2 listed after Example 3.

### EXAMPLE 3

PLA₂ Assay Using [³-H]-Oleic Acid Labeled E. coli

The procedure is a modification of that described by Elsbach and Weiss (1991). E. coli strain SJ198 was grown in 10 ml M9 minimal medium plus 50 μl glycerol, 100 μl vitamin mix, 40 μl methionine (25 mg/ml), 50 μl [³H]-oleic acid (0.1 μCi/μl), and 100 μl casamino acids (100 mg/ml). After incubation at 37° C. overnight, the culture was centrifuged and washed three times in PBS, autoclaved for 15 minutes, washed again three times in PBS and stored at −70° C. The reaction mixture contained 10 μl of 5×assay of the PLA₂ enzyme preparation, 10 μl of [³H]-oleic acid-labeled E. coli cells, and enough H₂O to adjust the total volume to 50 μl. The reaction mixture was incubated for 15 minutes at 37° C. in a waterbath. To stop the reaction, the tubes were transferred immediately to an ice bath and an equal volume of either 1 M HCl or 4 volumes cold PBS was added. After centrifugation in a microfuge at 4° C. for 10 minutes, an aliquot equal to 25% of the volume of the supernatant was used to measure the amount of [³H]-labeled oleic acid released from the E. coli cells by PLA₂ using liquid scintillation counting. Results of these assays are shown in Tables 1 and 2 below.

TABLE 1
Effect of melittin and PLAP peptides on the activity of PLA₂ from bee venom, snake
venom, bovine pancreas, and synovial fluid from rheumatoid arthritis patients, using
unsonicated vesicles labeled with [¹⁴C]-phosphatidylcholine (A) or
phosphatidylethanolamine (B) and [³H]-oleic acid-labeled E. coli as substrate (C&D).*
PLA₂ activity was expressed as μmoles/min/mg of protein in case of purified PLA₂
enzyme, while that in synovial fluid was expressed as μmole/min ml.
Phospholipase A₂ activity

| | | | | |
|---|---|---|---|---|
| Enzyme | | | | |
| source | No peptide | Melittin (1 μg) | G22R (10 μg) | G26 (10 μg) |

A

| | | | | |
|---|---|---|---|---|
| Bee V. | 5.63 ± 0.25 | 0.90 ± 0.08 | 3.15 ± 0.15 | 3.86 ± 0.15 |
| Snake V. | 18.81 ± 0.26 | 1.41 ± 0.84 | 13.06 ± 0.28 | 17.60 ± 0.73 |
| Bovine P. | 9.9 ± 0.3 × 10⁻⁵ | 3.6 ± 0.9 × 10⁻⁵ | N.D. | N.D. |
| Synovial F. | 3.61 ± 0.18 × 10⁻³ | 2.9 ± 0.0 × 10⁻⁴ | 2.87 ± 0.46 × 10⁻³ | 3.57 ± 0.04×10⁻³ |

B

| | | | | |
|---|---|---|---|---|
| Bee V. | 36.17 ± 1.24 | 19.66 ± 2.08 | 17.48 ± 1.84 | 28.82 ± 2.40 |
| Snake V. | 0.71 ± 0.08 | 0.05 ± 0.03 | 0.43 ± 0.04 | 0.61 ± 0.14 |
| Bovine P. | 5.21 ± 1.70 × 10⁻³ | 2.96 ± 0.0 × 10⁻³ | 3.68 ± 0.76 × 10⁻⁴ | 5.33 ± 1.46×10⁻⁴ |
| Synovial F. | 6.93 ± 0.25 × 10⁻³ | 4.7 ± 0.0 × 10⁻⁴ | 7.90 ± 1.56 × 10⁻³ | 7.03 ± 0.0×10⁻³ |

C

| | | | | |
|---|---|---|---|---|
| Bee V. | 989 ± 28.3 | 801 ± 55.0 | 942 ± 96.0 | 813 ± 69.0 |
| Snake V. | 143.47 ± 7.8 | 61.5 ± 2.75 | 147.5 ± 9.64 | 130.3 ± 8.78 |
| Bovine P. | 1.61 ± 0.01 | 0.06 ± 0.03 | 1.27 ± 0.25 | 1.59 ± 0.04 |
| Synovial F. | 73.7 ± 3.9 | 5.5 ± 0.4 | 73.3 ± 5.15 | 71.8 ± 3.31 |

D

| | | | | |
|---|---|---|---|---|
| Bee V. | 779.0 ± 40.0 | 1315.0 ± 48.2 | 745.0 ± 2.8 | 683.0 ± 48.2 |
| Snake V. | 46.01 ± 18.8 | 59.13 ± 13.97 | 102.7 ± 13.3 | 81.1 ± 0.85 |
| Bovine P. | 1.47 ± 0.27 | 1.51 ± 0.17 | 1.29 ± 0.13 | 1.44 ± 0.04 |
| Synovial F. | 29.09 ± 1.15 | 43.65 ± 3.19 | 25.86 ± 3.03 | 25.53 ± 5.6 |

*In section C, the reaction was stopped by diluting with four volumes of cold PBS, while in section D, the reaction was stopped by adding an equal volume of 1N Hcl.

TABLE 2
Effect of melittin (MLT) and its related peptides on the activity
of affinity-purified PLA₂ enzyme isolated from synovial fluid
of rheumatoid arthritis patients. The PLA₂ assay
consisted of unsonicated vesicles labeled with
[¹⁴C]-phosphatidylcholine. In each case the
mean is derived from three determinations.

| | | | | Phospholipase A₂ activity | (μ moles/ |
|---|---|---|---|---|---|
| | | | | B(× 10⁻³) | min/ml) C |
| | | | | [¹⁴C]- | [³H]-oleic |
| # | Peptide | Conc. | | phosphatidylcholine | acid-E. coli |
| 1 | — | — | | 0.27 ± 0.02 | 0.48 ± 0.02 |
| 2 | Melittin | 1.0 | μM | 0.055 ± 0.010 | 0.50 ± 0.12 |
| 3 | Melittin | 5.0 | μM | N.D. | 0.12 ± 0.02 |
| 4 | Melittin | 10.0 | μM | N.D. | 0.06 ± 0.03 |
| 5 | G22R | 10 | μM | 0.17 ± 0.01 | 0.27 ± 0.05 |
| 6 | G23 | 10 | μM | 0.24 ± 0.03 | 0.28 ± 0.07 |
| 7 | G24 | 10 | μM | 0.29 ± 0.01 | 0.39 ± 0.06 |
| 8 | G25 | 10 | μM | 0.14 ± 0.03 | 0.71 ± 0.06 |
| 9 | G26 | 10 | μM | 0.24 ± 0.04 | 0.41 ± 0.12 |
| 10 | G27 | 10 | μM | 0.21 ± 0.03 | 0.40 ± 0.02 |
| 11 | G141 | 10 | μM | 0.26 ± 0.04 | 0.37 ± 0.14 |
| 12 | G142 | 10 | μM | 0.21 ± 0.04 | 0.38 ± 0.21 |
| 13 | G141 + G142 | 5 + 5 | μM | 0.20 ± 0.03 | 0.42 ± 0.02 |
| 14 | F31 | 10 | μM | N.D. | 0.32 ± 0.04 |
| 15 | F32 | 10 | μM | N.D. | 0.38 ± 0.06 |

The order of inhibitory potency was found to be:

MLT>G25>G22>G141>G27>G23.

TABLE 2A
Inhibitory Potency

| | |
|---|---|
| MLT | 80% |
| G25 | 49% |
| G141 | 25% |
| G27 | 24% |

### EXAMPLE 4

Inhibitory Effect of Plap-Like Peptides on [³H]-Arachidonic Acid Release from Murine Monocyte/Macrophage Cells

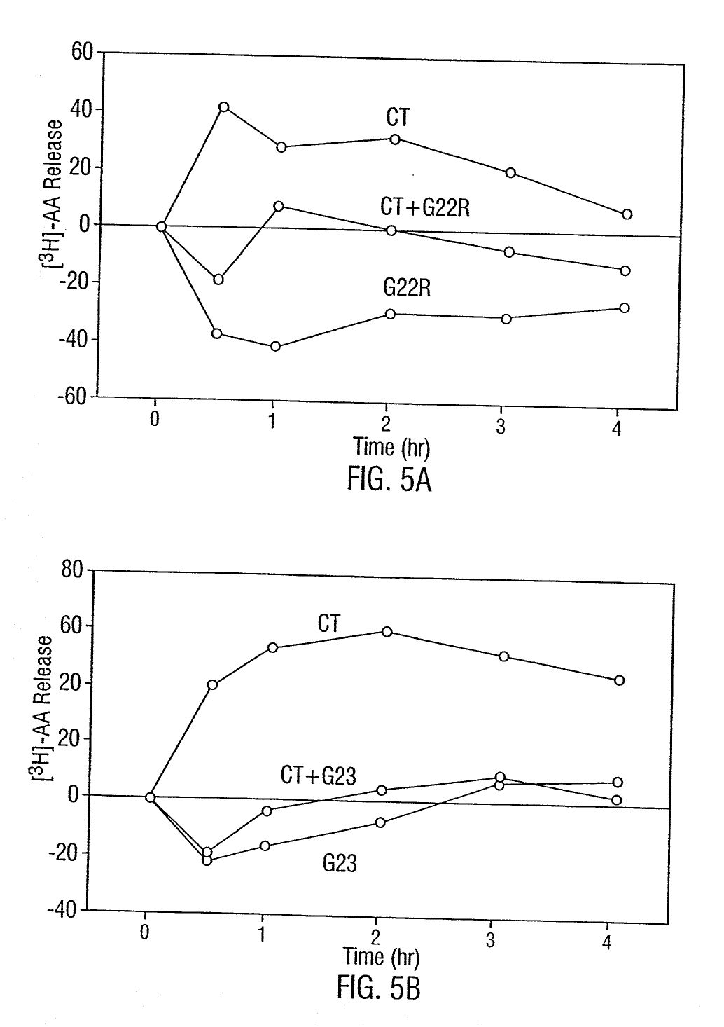

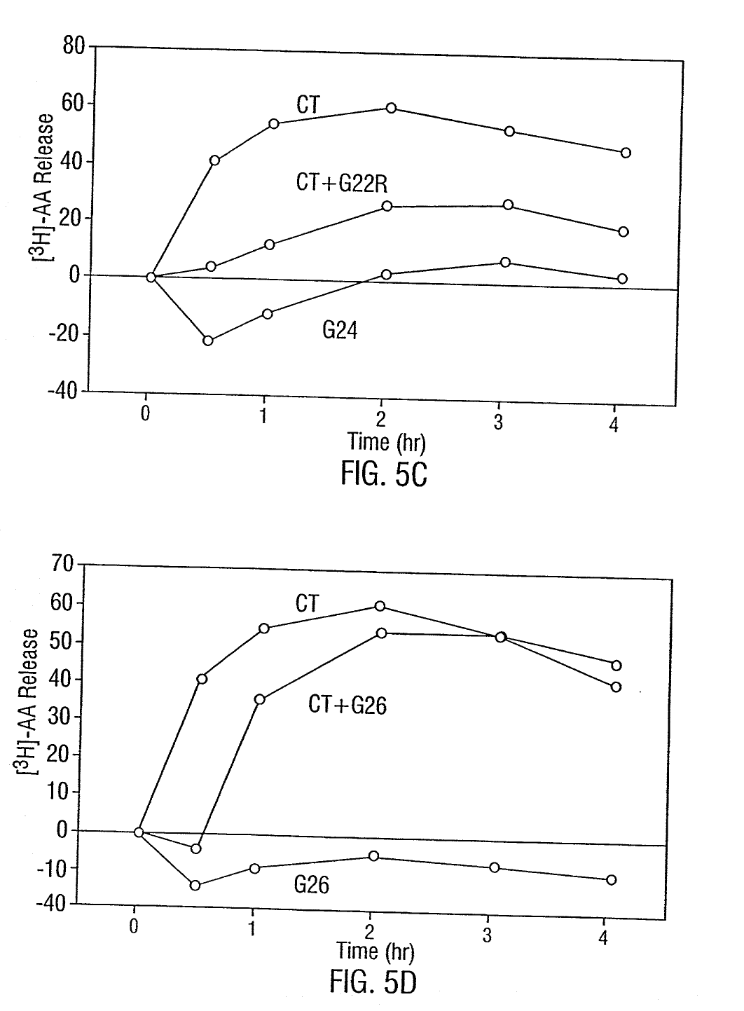

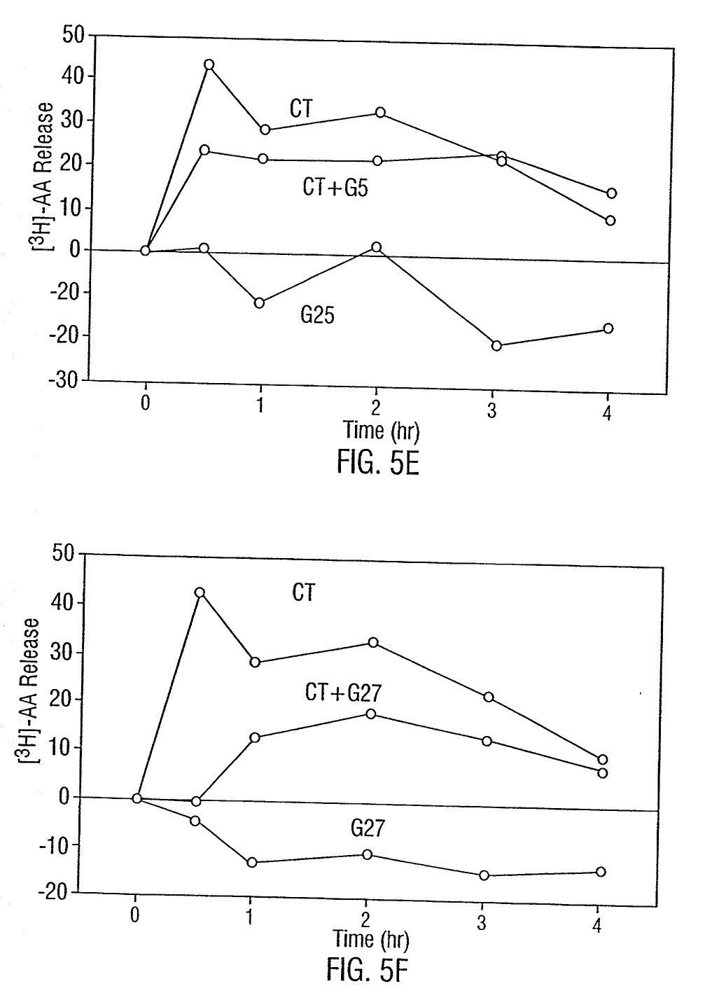

Further data concerning PLAP peptide inhibition of PLA₂ activity in murine monocyte/macrophage cells indicate that peptide-mediated inhibition of PLA₂ activity is possible within living cells, an important factor in determining the peptides usefulness as pharmaceuticals. The phospholipids in the cells were labeled prior to the assay with [³H]-archidonic acid. FIG. 5A, FIG. 5B, FIG. 5C, FIG. 5D, FIG. 5E, and FIG. 5F show that the synthetic peptides of this invention not only inhibit the amount of spontaneous [³ H]-arachidonic acid released from these cells, but also reduce the amount of [³H]-arachidonic acid released following stimulation of the cells with cholera toxin. Release of [³H]-arachidonic acid metabolites stimulated by cholera toxin (CT) has previously been shown [Reitmeyer and Peterson (1990) The authors described the general technique of labeling eukaryotic cells with [³H]-arachidonic acid by adding 0.3-1 μCi of the radiolabeled fatty acid to the tissue culture media. After incubating overnight at 37° C. with 5% CO₂, the cells were washed (3×) with medium. The labeled cells were then stimulated with cholera toxin, and the amount of [³H]-arachidonic acid metabolites released with 2-4hours was assessed by liquid scintillation counting. Comparisons were made with the controls.

### EXAMPLE 5

Microtitter Assay of Direct Binding of PLA₂ and Plap-Like Peptides

Each well of a polypropylene ELISA plate was coated with either 100 μl of bee venom PLA₂ (10 μg/ml) or synovial fluid (1:10 diluted) diluted in 0.1 M sodium carbonate (pH 9.6) overnight. After washing with PBS-Tween and blocking with 10% BSA, the wells of the plate were treated with serial dilutions of G22B for 120 minutes. The plates were then washed and 100 μl 2.5 μg/mL streptavidin-peroxidase conjugate was added to each well. After incubation with the streptavidin-peroxidase for 60 minutes, the plates were again washed and each well was developed with 100 μl of 3.3 mg/mL ABTS substrate for 30 minutes. Absorbance was read at 405 nm. Results are shown in FIG. 6A.

### EXAMPLE 6

Interference of Streptavidin-Peroxidase Binding to Biotin-Mellitin by Synovial Fluid PLA₂

Each well of a polypropylene microtiter ELISA plate was coated with 100 μl of 1 μM biotinylated synthetic melittin overnight at room temperature The dried plate was washed with PBS containing, 0.5% Tween 20 and blocked with 10% BSA. Serial dilutions of synovial fluid were added to the wells for 120 minutes. After washing the plates with PBS, 100 μl of 2.5 μg/mL streptavidin-peroxidase was added to each well for 60 minutes. The plates were again washed and 100 μl of 3.3 mg/mL ABTS substrate was added After 30 minutes, absorbance was read at 405 nm. The results are shown in FIG. 6B.

### EXAMPLE 7

Affinity Chromatography with Plap-Like Peptides

The peptides of the present invention may be conjugated to various solid matrices. One preferred solid matrice is epoxy-activated Sepharose 6B(Pharmacia Biotech Uppsala Sweden). Columns can be packed with slurries of such material mixed with buffers, then a solution containing PLA₂ can be allowed to run through the column. The column can be washed with PBS so that all of the non-PLA₂ components are eluted. Quantitation of PLA₂ eluted from the column by low pH buffer (2.8) can be accomplished.

Conjugation of Peptides with epoxy-activated Sepharose: Synthetic melittin was conjugated to Epoxy-activated Sepharose 6B (Pharmacia Biotech Uppsala Sweden) as follows: 10 g of Epoxy-activated Sepharose 6B was washed with 2.0 L of H₂O and 1.5 L of sodium carbonate buffer (0.1 M, pH 10.8). Synthetic melittin as well as PLAP peptide (5 mg each) were dissolved in distilled H₂O (2 ml), and was added to the gel slurry (5 ml for melittin and 10 ml for PLAP) and final volume of slurry was made to 30 ml with sodium carbonate buffer (0.1 M, pH 10.8) and allowed to shake overnight at room temperature. Unreacted sites were blocked with ethanolamine (1.0M, pH 8.0) by incubating at 43° C. for 3 hr and than transferring to a shaker at room temperature overnight One wash was given with ethanolamine prior to incubation at 43° C. The gel was washed 3 times with alternative cycles of Tris-HCl buffer (100 mM containing 500 mM NaCl, pH 8.0) and sodium acetate buffer (100 mM containing 500 NaCl, pH 4.0) The slurry was washed 3 times with sodium phosphate buffer (10 mM containing 150 mM NaCl, pH 7.0) and packed into the column.

Isolation of PLA₂ from human serum: 10 ml of heat-inactivated human serum Gemini Bio Product Inc. (Calabasas, Calif.) was diluted 1:10 with PBS and loaded onto Melittin-Sepharose as well as PLAP-Sepharose columns pre-equilibrated with PBS. Columns were washed with 500 ml of PBS overnight and eluted with 0.5M glycine/HCl buffer, pH 2.8 and 2 ml fractions were collected, neutralized to pH 7.2 with Tris-HCl buffer and analyzed for PLA₂ activity using [³H]labeled oleic acid—E. coli cells as substrate. Eluted fractions were concentrated and dialyzed in centricon 10 (Amicon, Inc., Beverly, Mass.) and the pass through was discarded. The retentate was again passed through centricon 30 and concentrated to about ⅕th of the volume. Both retentate and pass through were subjected to PLA₂, assay with and without melittin.

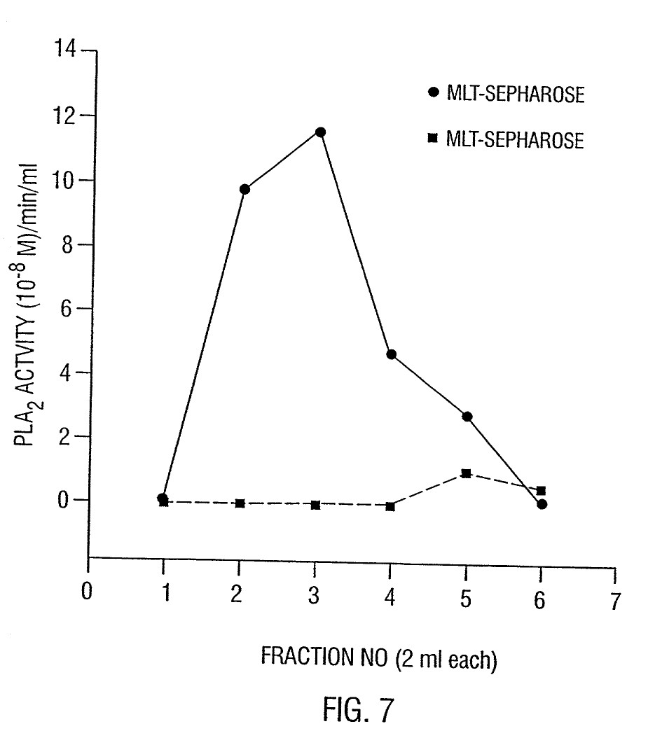

Results: When human serum is passed through a melittin-Sepharose column and the washed column is eluted with glycine buffer, PLA₂ activity was detected in fraction number 2, 3, 4, and 5, being maximum in fraction 3. However, chromatography through a PLAP-Sepharose column showed very little PLA₂ activity (FIG. 7). The fractions after concentration by centricon 10 or 30, showed a 4 fold reduction in PLA₂ activity in the presence of melittin. (Table 3). The principle of using a melittin-Sepharose affinity column to purify PLA₂ from normal human serum is illustrated in Table 4. In this experiment the specific activity of human serum PLA₂ was increased more than 1500 fold by this single step purification step, which illustrates the interaction between PLA₂ and melittin.

TABLE 3
Effect of Melittin (5 μM) on PLA₂ activity of Centricon
concentrated/pass through samples from human serum eluted from
MLT-Sepharose column (FIG. 7). [³H] - oleic
acid-labeled E. coli cells were used as substrate.

| | | PLA₂ activity (nano mol/min/ml) | |
|---|---|---|---|
| Sr# | Samples | Control | MLT |
| 1 | Human serum PLA₂ eluted | 812 ± 18.2 | 219 ± 16.3 |
| | from MLT-Sepharose column | | |
| 2 | as 1 but Centricon 30 | 696 ± 9.81 | 192 ± 5.44 |
| | concentrated (5 times) | | |
| 3 | as 1 but Centricon 30 pass through. | 197 ± 13.4 | 172 ± 8.73 |

FIG. 7 shows chromatography of human serum on peptide—Sepharose columns: 10 ml of heat inactivated human serum was diluted to 1:10 with PBS and loaded onto a melittin-Sepharose column (●) and PLAP—Sepharose column (▪) pre-equilibrated with PBS. Columns were washed with 500 ml of PBS for overnight and eluted with 0.5M glycine/HCl buffer, pH 2.8 and 2 ml fractions were collected, neutralized to pH 7.2 with Tris-HCl buffer and analyzed for PLA₂ activity using [3H] labeled oleic acid—E. coli cell as substrate.

TABLE 4
PLA₂ activity of human serum chromatographed on
melittin-Sepharose as determined by [³H]-oleic
acid-labeled E. coli cells. PLA₂ activity
expressed as units, where I unit = 1 μmol/min/mg of protein.

| Sr# | Source | PLA₂ activity (Units) | Purification |
|---|---|---|---|
| 1 | Human serum | 17.8 ± 1.4 | 0 |
| 2 | Human serum chromato- | 27066.6 ± 606.0 | 1521 fold |
| | graphed on melittin-Sepharose | | |

All of the compositions and/or methods disclosed and claimed herein can be made and executed without undue experimentation in light of the present disclosure. Wile the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

Abou-Samara, et al, “Role of arachidonic acid in the regulation of adrenocorticotropin release from anterior pituitary cultures,” Endocrinology, 119:1427-1431. 1986.

Argiolas and Pisano, “Facilitation of phospholipase A₂ activity by mastoparans, a new class of mast cell degranulating peptides from wasp venom,”]J. Biol. Chem., 258:13697-702, 1983.

Billingham, et al., “An anti-inflammatory peptide from bee venom,” Nature, 245:163-164, 1973.

Bomalaski et al., U.S. Pat. No. 5,294,698, Nov. 15, 1994.

Chang and Bliven, “Anti-arthritic effect of bee venom,” Agents & Actions, 9:205-211, 1979.

Choi, et al., “Effect of the amphipathic peptides melittin and mastopranon on calcium influx, phosphoinoside breakdown and arachidonic acid release in rat pheochromacytoma PC 12 cells,” J. Pharmac. Esp. Ther., 260:369-375, 1992.

Clark, et at, “Cloning of a phospholipase A₂-activating protein,” Proc. Natl. Acad. Sci. USA, 88:5418-5422, 1991.

Cuppoletti and Abbott, “Interaction of melittin with the (Na⁺-K⁺)ATPase: Evidence for a melittin-induced conformational change,” Arch. Biochem. Biophys., 283:249-257, 1990.

Cuppoletti and Malinowska, “Interaction of polypeptides with the gastric (H⁺-K⁺)ATPase: melittin, synthetic analogs, and a potential intracellular regulatory protein,”]Mol. Cell. Biochem., 114:57-63, 1992.

Cuppoletti, et al., “Melittin inhibition of the gastric (H⁺-K⁺) ATPase and photoaffinity labeling with [¹²⁴I]azidosalicylyl melittin,” Arch Biochem. Biophys.. 275:263-270, 1989.

Demartis, “Report on a Patient,” L'abeille Med, Paris, 30:233, 1859.

Dixon and Webb, “Enzyme Inhibitors,” Enzymes, Academic Press, New York, 315-359, 1964.

Dufourcq, et al., “Morphological changes of phosphatidylcholine bilayers induced by melittin, vesicularization, fusion, and discoidal particles,” Biochem. Biophys. Acta, 859:3348, 1986.

Eiseman, et al., “Effect of honeybee (Apis mellifera) venom on the course of adjuvant-induced arthritis and depression of drug metabolism in the rat,” Biochem. Pharm., 31:1139-1146, 1982.

Elsbach, et al., “Separation and purification of a potent bactericidal/permeability-increasing protein and a closely associated phospholipase A2 from rabbit polymorphonuclear leukocytes. Observations on their relationship”, J. Biol. Chem., 254:11000-11009, 1979.

Engelsen and Zatz, “Stimulation of fatty acid methylation in human red cell membranes by phospholipase A₂ activation,” Biochem Biophys. Acta, 711:515-520, 1982.

Fletcher, et al, “Contribution of bee venom phospholipase A₂ contamination in melittin fractions to presumed activation of tissue phospholipase A₂,” Toxicon, 28(6):647-56, 1990.

Grandison, “Stimulation of anterior pituitary prolactin release by melittin, an activator of phospholipase A₂,” Endocrinology, 114:1-7, 1984.

Haberman, “Bee and wasp venoms,” Science, 177:314-322,1972.

Hazlett and Dennis, “Affinity chromatography of phospholipase A2 from Naja naja naja (Indian cobra) venom,” Toxicon, 23:457-466, 1985.

Hessler, “Phospholipase A₂ activation by melittin causes amylase release from exocrine pancreas,” Can. J. Physiol. Pharm, 67:411-416, 1989.

Knepel and Gerhards, “Stimulation by melittin of adrenocorticotropin and beta-endorphin from rat adenohypophysis in vitro,” Prostaglandins, 33:479-490, 1987.

Kortesuo, et al., “Rat pancreatic phospholipase A2. Purification, localization and development of an enzyme immunoassay,” Int'l. J. Pancreatology, 13:111-118, April 1993.

Liu and Jackson, “Differential actions of arachidonic acid and melittin on luteinizing hormone release and synthesis,” Neuroendocrin., 49:617-622, 1989.

Metz, “Lack of specificity of melittin as a probe of insulin release mediated by endogenous phospholipase A₂ or lipoxygenase,” Biochem. Pharmacol., 35:3371-3381, 1986.

Mollay and Kreil, “Enhancement of bee venom phospholipase A₂ activity by melittin, direct lytic factor from cobra venom and polymyxin B,” FEBS Lett., 46:141-144, 1974.

Mollay, et al., “Action of phospholipases on the cytoplasmic membrane of Escherichia coli. Stimulation by melittin,” Biochim. Biophys. Acta, 426:317-324, 1976.

Okano, et al., “A wasp venom mastoporan-induced polyphosphoinositide breakdown in rat peritoneal mast cells,” FEBS Lett., 188:363-366, 1985.

Osol and Farrar, U.S. Dispensatory, Philadelphia, J. B. Lippinott Co. (25th ed.), 1598-1599, 1955.

Peterson, et al, “Cholera toxin induces synthesis of phospholipase A₂-activating protein,” Infect. Immun., 64:2137-2143, 1996.

Peterson, et al., “Phospholipase A₂-activating protein and idiopathic inflammatory bowel disease,” Gut., 39:698-704, 1997.

Rao, “Differential susceptibility of phosphatidylcholine small unilamellar vesicles to phospholipases A₂, C and D in the presence of membrane active peptides,” Biochem. Biophys. Res. Comm., 182:682-688, 1992.

Raynor, et al., “Membrane interactions of amphiphilic polypeptides mastoparan, melittin, polymyxin B, and cardiotoxin. Differential inhibition of protein kinase C, Ca²⁺/calmodulin-dependent protein kinase II and synaptosomal membrane Na⁺,K⁺-ATPase, and Na⁺ pump and differentiation of HL60 cells,” J. Biol. Chem, 266:2753-2758, 1991.

Reitmeyer and Peterson, “Stimulatory effects of cholera toxin on arachidonate metabolism in Chinese hamster ovary cells,” Proc. Soc. Exp. Biol. Med., 193:181-184, 1990.

Rosenthal and Jones, “Release of arachidonic acid from vascular endothelial cells: Fatty acyl specificity is observed with receptor-mediated agonists and with the calcium ionophore A23187 but not with melittin,” J. Cell. Physiol, 136:333-340, 1988.

Salari, et al, “Stimulation of lipoxygenase product synthesis in human leukocytes and platelets by melittin,” Mol. Pharmacol., 23:546-548, 1985.

Segel, “How to solve mathematical problems in general biochemistry,” Biochemical Calculations, Wiley, N.Y. (2nd ed.), 1976.

Shier, “Activation of high levels of endogenous phospholipase A₂ in cultured cells,” Proc. Natl. Acad. Sci. USA, 76:195-199, 1979.

Somerfield and Brandwein, “Bee venom and adjuvant arthritis [letter],” J. Rheum., 15:1878, 1988.

Somerfield, “Bee venom and adjuvant arthritis [letter],” J. Rheum, 10:522, 1983.

Somerfield, “Bee venom and arthritis [letter],” J. Rheum., 13:477, 1986.

Somerfield, “Bee venom and arthritis: magic, myth or medicine,” New Zealand Med. J., 99:281-283, 1986.

Somerfield, et al., “Bee venom melittin blocks neutrophil O₂-production,” Inflammation, 10:175-182,1986.

Stefanski, et al., “Purification of a soluble phospholipase A₂ from synovial fluid in rheumatoid arthritis,” J. Biochem., 100:1297-1303, 1986.

Steigerwaldt, et al, “Standardized bee venom (SBV) therapy of arthritis. Controlled study of 50 cases with 84 percent benefit,” Industrial Medicine & Surgery, 35:1045-9, 1966.

Tannenbaum and Greenspoon, “Bee venom therapy of adjuvant arthritis [letter],” J. Rheum., 9:649, 1982.

Verheij, et al., “Structure and function of phospholipase A₂,” {Review] Rev. Physiol. Biochem. Pharmacol., 91.91-203, 1981.

Voss, et al., “Effects of melittin on molecular dynamics and Ca⁺⁺-ATPase activity in sarcoplasmic reticulum membranes: time-resolved optical anisotropy,” Biochem., 30:7498-7506, 1991.

Wang, et al, “Cloning of a rat cDNA encoding a protein with high homology TO mouse phospholipase A₂-activating protein,” Gene, 161:237-241, 1995.

Yiangou, et al., “Modulation of alpha I-acid glycoprotein (AGP) gene induction following honey bee venom administration to adjuvant arthritic (AA) rates; possible role of AGP on AA development,” Clin. & Exper. Immun., 94:156-62, 1993.

Yunes, et al., “Phospholipases: Melittin facilitation of bee venom phospholipase A₂-catalyzed hydrolysis of unsonicated lecithin liposomes,” Arch. Biochem. Biophys., 183:105-112, 1977.

Zurier, et al., “Effect of bee venom on experimental arthritis,” Annals of the Rheum. Dis., 32:466-470, 1973.

1. Synthetic melittin, peptide G22R, peptide G23, peptide G24, peptide G25, peptide G26, peptide G27, peptide G141, peptide G142, peptide F31, or peptide F32 usable for the treatment of a disease characterized by a undesirable inflammatory response.

2. The composition of claim 1 wherein the disease is rheumatoid arthritis, tuberculosis, asthma, burn wounds, inflammation of the gastrointestinal tract, or inflammation caused by an infectious disease.

3. A method of treating a disease characterized by an undesired inflammatory response comprising administering a composition containing synthetic melittin, peptide G22R, peptide G23, peptide G24, peptide G25, peptide G26, peptide G27, peptide G141, peptide G142, peptide F31, or peptide F32 at an appropriate dosage.

4. A method of purifying PLA₂ using a composition containing synthetic melittin peptide G22R, peptide G23, peptide G24, peptide G25, peptide G26, peptide G27, peptide G141, peptide G142, peptide F31, or peptide F32.

5. A method of diagnosing a disease characterized by the presence of PLA₂ comprising detecting PLA₂ with compositions containing synthetic melittin peptide G22R, peptide G23, peptide G24, peptide G25, peptide G26, peptide G27, peptide G141, peptide G142, peptide F31, or peptide F32.

6. The method of claim 5 where the disease is rheumatoid arthritis.

7. A diagnostic test based on detection of the unique amino acid sequence of human PLAP.

8. A polynucleotide having SEQ ID No. 1.

9. A protein coded by the polynucleotide of SEQ ID No. 1.

10. A peptide having SEQ ID No. 2.

11. A peptide having SEQ ID No. 3

12. A peptide having SEQ ID No. 4.

13. A peptide having SEQ ID No. 5.

14. A peptide having SEQ ID No. 6.

15. A peptide having SEQ ID No. 7.

16. A peptide having SEQ ID No. 8.

17. A peptide having SEQ ID No. 9.

18. A peptide having SEQ ID No. 10.

19. A peptide having SEQ ID No. 11.

20. A peptide having SEQ ID No. 12.

21. A peptide having SEQ ID No. 13.
